(19) United States
(12) Patent Application Publication
(10) Pub. No.: US 2023/0149617 A1
(43) Pub. Date: May 18, 2023

(54) METHODS OF TREATING CONDITIONS

(71) Applicant: NATIONAL JEWISH HEALTH, Denver, CO (US)

(72) Inventor: Glenn A. HIRSCH, Denver, CO (US)

(21) Appl. No.: 17/915,907

(22) PCT Filed: Apr. 2, 2021
(86) PCT No.: PCT/US2021/025602

§ 371 Date: Sep. 29, 2022

Related U.S. Application Data
(60) Provisional application No. 63/005,125, filed on Apr. 3, 2020.
(60) Provisional application No. 63/019,675, filed on May 4, 2020.

(51) Int. Cl. A61M 1/36; A61P 31/16; A61N 5/06

(52) U.S. Cl. CPC A61M 1/3683; A61N 5/06; A61P 31/16; A61N 2005/0661

(57) ABSTRACT

The present disclosure relates to a method of treating conditions and/or diseases using blood irradiation. Conditions and diseases include cytokine storm syndrome, autoimmune diseases, and infectious diseases. The disclosure also relates to modulation of the immune system, for example by modulating leukocytes. The method involves removal of blood, exposing the removed blood to radiation, and placing the exposed blood into a patient. Radiation includes ultraviolet radiation and other types of radiation.

### CROSS REFERENCE TO RELATED APPLICATIONS

This application claims the benefit of U.S. Provisional Pat. Application No. 63/005,125, filed Apr. 3, 2020, and U.S. Provisional Pat. Application No. 63/019,675, filed May 4, 2020, the disclosures of which are incorporated herein by reference in their entirety.

### FIELD

The present disclosure relates to a method of treating conditions and/or diseases using blood irradiation. The present disclosure further relates to blood irradiation for the treatment of cytokine storm syndrome, autoimmune diseases, and infectious diseases, for modulation of the immune system, and for other purposes.

### BACKGROUND

Currently, there is a great need for the development of new treatments that are effective against infections, particularly against viral infections which are associated with high morbidity and mortality, and which impact on sizable populations. Treatments currently available are inadequate or ineffective in large proportions of infected patients.

A well-known family of pathogenic viruses are the Coronaviruses. Coronaviruses (Order Nidovirales, family Coronaviridae, Genus Coronavirus) are enveloped positive-stranded RNA viruses that bud from the endoplasmic reticulum-Golgi intermediate compartment or the cis-Golgi network (Fischer, Stegen et al. 1998; Maeda, Maeda et al. 1999; Corse and Machamer 2000; Maeda, Repass et al. 2001; Kuo and Masters 2003). Coronaviruses infect humans and animals and it is thought that there could be a coronavirus that infects every animal. Per the Center for Disease Control and Prevention (CDC), coronaviruses are named for the crown-like spikes on their surface and comprise four main sub-groupings known as alpha, beta, gamma, and delta., examples of which include: 229E (alpha coronavirus), NL63 (alpha coronavirus), OC43 (beta coronavirus), HKU1 (beta coronavirus)). Other human coronaviruses are MERS-CoV (the beta coronavirus that causes Middle East Respiratory Syndrome, or MERS), SARS-CoV (the beta coronavirus that causes severe acute respiratory syndrome, or SARS), and SARS-CoV-2 (the novel coronavirus that causes coronavirus disease 2019, or COVID-19). People around the world commonly get infected with human coronaviruses 229E, NL63, OC43, and HKU1. Sometimes coronaviruses that infect animals can evolve and make people sick and become a new human coronavirus. Three recent examples of this are SARS-CoV-2, SARS-CoV, and MERS-CoV. (cdc.gov/coronavirus/types.html; February 2020).

Two human coronaviruses, 229E and OC43, are known to be the major causes of the common cold and can occasionally cause pneumonia in older adults, neonates, or immunocompromised patients (Peiris, Lai et al. 2003). Animal coronaviruses can cause respiratory, gastrointestinal, neurological, or hepatic diseases in their host (Peiris, Lai et al. 2003). Several animal coronaviruses are significant veterinary pathogens (Rota, Oberste et al. 2003).

Although coronaviruses were first identified decades ago, they only received notoriety in 2003 when one of their members was identified as the etiological agent of severe acute respiratory syndrome (SARS). Since then, MERS-CoV and SARS-CoV-2 have become substantial health concerns, with SARS-CoV-2 causing a global pandemic that began in 2019. Previously these viruses were known mainly to be important agents of respiratory and enteric infections of domestic and companion animals and to cause approximately 15% of all cases of the common cold.

In general, coronaviruses are well known and most of those who are diagnosed with it recover completely with no complications after receiving the needed supportive therapy. However, in some of the patients who are infected, serious complications can develop affecting the respiratory system, immune system, organs, and other body systems. Coronaviruses can cause death, especially among the elderly, in patients with chronic respiratory and/or cardiac conditions, and in immunocompromised patients.

To improve the prospect of treating and preventing viral infections, such as coronavirus infections, there is an on-going need to identify treatments capable of mitigating various aspects of respiratory viral infection.

### SUMMARY

The present disclosure generally relates to a method for the therapeutic or prophylactic treatment of a subject patient having or being at risk of cytokine storm syndrome or other diseases by the extracorporeal administration of blood irradiation (BI) to blood of a patient or other donor.

In some embodiments, the invention relates to a method of treating a patient having or being at risk of cytokine storm syndrome. The treatment involves removing a portion of blood from the patient, exposing the portion of blood to ultraviolet radiation, and replacing the exposed portion of blood into the patient. The treatment improves a treatment outcome of the cytokine storm syndrome.

In some embodiments, the invention relates to a method of treating a patient having or being at risk of cytokine storm syndrome. The treatment involves removing a portion of blood from a donor, exposing the portion of blood to ultraviolet radiation, and placing the exposed portion of blood into the patient. The treatment improves a treatment outcome of the cytokine storm syndrome.

In some embodiments, the invention relates to a method of treating a patient having or being at risk of autoimmune disease. The treatment involves removing a portion of blood from a donor, exposing the portion of blood to ultraviolet radiation, and placing the exposed portion of blood into the patient. The treatment improves a treatment outcome of autoimmune disease.

In some embodiments, the invention relates to a method of treating a patient having or being at risk of an infectious disease. The treatment involves removing a portion of blood from a donor, exposing the portion of blood to ultraviolet radiation, and placing the exposed portion of blood into the patient. In some aspects, the patient develops an immune response against the infectious disease and/or the causative agent of the infectious disease.

In some embodiments, the invention relates to a method of modulating leukocytes in a patient. The treatment involves removing a portion of blood from a donor, exposing the portion of blood to ultraviolet radiation, and placing the exposed portion of blood into the patient. The treatment improves a treatment outcome of a disease.

In some embodiments, the invention relates to a method of treating a patient having or being at risk of COVID-19. The treatment involves removing a portion of blood from a donor, exposing the portion of blood to ultraviolet radiation, and placing the exposed portion of blood into the patient. The treatment improves a treatment outcome of COVID-19.

In some embodiments, the patient has, had, or is at risk of cytokine storm syndrome.

In some aspects, the cytokine storm syndrome is associated with a condition selected from the group consisting of viral, bacterial, parasitic or fungal infection, acute respiratory distress syndrome, trauma, reaction to a blood transfusion, reaction to a medication, graft vs. host disease, gastric aspiration, sepsis, hemophagocytic lymphohistiocytosis, autoimmune disease, and combinations.

In some aspects, the condition is a respiratory viral infection by a virus selected from the group consisting of influenza virus, coronavirus, hantavirus, cytomegalovirus, herpesvirus, varicella virus, rhinovirus, parainfluenza virus, human metapneumovirus, adenovirus, respiratory syncytial virus, Ebstein-Barr virus, Dengue virus, West Nile virus, Western and Eastern equine encephalitis viruses, polio, chikungunya variola virus, Ebola virus, and orthopoxvirus. In some aspects, the respiratory viral infection is infection by a coronavirus, which in some aspects is SARS-CoV-2.

In certain embodiments, the step of removing involves removing between about 0.1% and about 20%, between about 1% and about 20%, or between about 5% and about 15% of the blood of the patient. In certain aspects, between about 1.0 and about 5.0 milliliters of blood are removed per kilogram of body weight of the patient.

The type of ultraviolet radiation is, in some embodiments, UVA, UVB, UVC, near ultraviolet, middle ultraviolet, far ultraviolet, hydrogen Lyman-alpha, vacuum ultraviolet, or extreme ultraviolet. In some aspects, the ultraviolet radiation is UVA, UVB, and/or UVC.

In some aspects, the radiation is ionizing radiation. In some aspects, the radiation is molecularly damaging radiation. In certain aspects, the radiation is particle radiation, and, in some aspects, the particle radiation is selected from the group consisting of alpha radiation, beta radiation, neutron radiation, positron radiation, and proton radiation. In certain aspects, the radiation is electromagnetic radiation, and, in some aspects, the radiation has a wavelength less than about 380 nm. In some aspects, the type of radiation is ultraviolet radiation selected from the group of UVA, UVB, UVC, near ultraviolet, middle ultraviolet, far ultraviolet, hydrogen Lyman-alpha, vacuum ultraviolet, extreme ultraviolet, and combinations thereof, and, in certain aspects the radiation is ultraviolet radiation selected from the group of UVA, UVB, UVC, and combinations thereof. In certain aspects, the radiation has a wavelength less than about 10 nm, and, in some aspects, the radiation has a wavelength less than about 10 pm. In some aspects, the radiation comprises γ-rays.

In some embodiments, the blood is exposed to radiation at a dosage of a dosage sufficient to elicit an improved treatment outcome.

In some embodiments, the treatment outcome is selected from the group consisting of reduced viral load, reduced risk of cytokine storm syndrome, reduced severity of cytokine storm syndrome, reduced risk of sepsis, reduced severity of sepsis, and reduced severity of autoimmune disease. In certain aspects, the treatment outcome is reduced viral load. In some aspects, the viral load in the portion of blood is reduced by about 100%, at least about 99%, at least about 95%, at least about 90%, at least about 80%, at least about 70%, at least about 60%, at least about 50%, at least about 40%, at least about 30%, at least about 20%, or at least about 10%. In some aspects, the treatment outcome is risk of cytokine storm syndrome. In some aspects, the risk of cytokine storm syndrome in the patient after placing the exposed portion of blood into the individual is reduced by about 100%, by at least about 99%, by at least about 95%, by at least about 90%, by at least about 80%, by at least about 70%, by at least about 60%, by at least about 50%, by at least about 40%, by at least about 30%, by at least about 20%, or by at least about 10%. In some aspects, the severity of cytokine storm syndrome is determined by blood levels of a protein selected from the group consisting of an interleukin, a chemokine, a colony-stimulating factor, a tumor necrosis factor, and combinations thereof. The severity of cytokine storm syndrome is, in some aspects, determined by blood levels of IL-2, IL2R, IL-6, IL-7, IL-8, IL-10, IL-1β, IL-1RA, IL-12, IL-17, TNF-α, GSCF, IP10, MCP1/CCL2, CCL5, CXCL9, IP-10/CXCL10, MIG protein/CXCL9, MIP1A, CSF3R, IFNγ, IFNAR1, IFNGR1, CD58,TNFα, and combinations thereof. In some aspects, the severity of cytokine release is determined by absence of or deceased severity of a symptom. Such symptoms are, in some aspects, lung injury, fever, swelling, edema, redness, fatigue, nausea, and/or sepsis.

In certain embodiments, the treatment outcome is severity of autoimmune disease. In some aspects, the severity of autoimmune disease is determined by absence of or deceased severity of a symptom. Such symptoms include fatigue, joint pain, joint swelling, skin problems, vascular inflammation, vascular problems, myocarditis, arrhythmias, nerve problems, abdominal pain, digestive problems, fever, and/or swollen glands.

In some embodiments, the patient has or had a condition selected from the group consisting of viral, bacterial, parasitic or fungal infection, acute respiratory distress syndrome, trauma, reaction to a blood transfusion, reaction to a medication, graft vs. host disease, gastric aspiration, sepsis, hemophagocytic lymphohistiocytosis, autoimmune disease, and combinations thereof. In some embodiments, the donor has or had a condition selected from the group consisting of viral, bacterial, parasitic or fungal infection, acute respiratory distress syndrome, trauma, reaction to a blood transfusion, reaction to a medication, graft vs. host disease, gastric aspiration, sepsis, hemophagocytic lymphohistiocytosis, autoimmune disease, and combinations thereof. In some embodiments the condition is an infection by an unknown microorganism or unknown types of microorganism.

In some embodiments, the autoimmune disease is selected from the group consisting of Crohn’s disease, eczema, psoriasis, psoriatic arthritis, rheumatoid arthritis, systemic lupus erythematosus, sarcoidosis, granulomatosis with polyangiitis, myocarditis, inflammatory bowel disease, multiple sclerosis, type 1 diabetes mellitus, Guillain-Barre syndrome, chronic inflammatory demyelinating polyneuropathy, Graves’s disease, and myasthenia graves

Some embodiments include further treating the portion of blood. The further treatment in some aspects involves contacting the blood with a compound selected riboflavin, psoralens, amotosalen HCl, porphyrins, photosensitizers, chlorophyll derivatives, light-sensitive agents, UV-activated nucleic acid replication inhibitors, dyes (such as such as neutral red, methylene blue, acridine, toluidines, flavine (acriflavine hydrochloride) and phenothiazine derivatives), coumarins, quinolones, quinones, and anthroquinones.

In some embodiments, the methods cause downregulation of the patient’s immune system. In some aspects, the downregulation of the patient’s immune system is caused by inactivating, attenuating, or downregulating white blood cell activity of the patient. In some aspects, the downregulation of the patient’s immune system is caused by inactivating, attenuating, or downregulating white blood cells in the portion of blood. In certain aspects, the downregulation of the patient’s immune system is caused by inactivating, attenuating, or downregulating deleterious cytokine activity in the portion of blood and/or in the patient.

In some embodiments, the leukocytes are selected from the group consisting of neutrophils, eosinophils, basophils, macrophages, B cells, and T cells. In some aspects, the T cells are selected from the group consisting of CD4+ T cells, CD8+ T cells, helper T cells, cytotoxic T cells, memory T cells, regulatory T cells, natural killer T cells, mucosal associated invariant T cells, and gamma delta T cells.

In some embodiments, there is a vaccine-like effect caused by embodiments of the invention. In some aspects, the radiation creates antigens in the portion of blood. In certain embodiments, the antigens induce an immune response in the patient against an infectious agent.

In some embodiments, the patient is the donor.

In some embodiments, the patient has, had, or is at risk of having an infection caused by at least one multi-drug resistant organism. In some embodiments, the donor has, had, or is at risk of having an infection caused by at least one multi-drug resistant organism.

### DETAILED DESCRIPTION

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

All publications, patents and published patent applications referred to in this application are specifically incorporated by reference herein. In case of conflict, the present specification, including its specific definitions, will control.

Respiratory viral infections are a major cause of illness and mortality, and respiratory viral infections are a major cause of hospitalization. The types of respiratory viral infection contemplated by the invention are not limited to infections caused by specific types of virus, e.g. to a specific family, genus, or species of virus. Examples of viruses contemplated by the invention are influenza virus, coronavirus, hantavirus, cytomegalovirus, herpesvirus, and orthopoxvirus.

Types of influenza include but are not limited to Influenza A, Influenza B, Influenza C, Influenza D and could include other types of influenza that do not yet exist or that have not been described or discovered. There are many types of Influenza A, which include but are not limited to H1N1, H2N2, H3N2, H5N1, H7N7, H1N2, H9N2, H7N2, H7N3, H10N7, H7N9, and H6N1. The naming conventions for influenza are well known in the art, and a person of skill in the art would know how new types of influenza would be named.

SARS-CoV-2 is an example of a coronavirus that frequently causes severe symptoms and death. SARS-CoV-2 infection causes the disease COVID-19, which is characterized by numerous respiratory and non-respiratory symptoms. Symptoms include cough, fever, chills, shortness of breath, difficulty breathing, muscle aches, body aches, loss of taste and/or smell, diarrhea, headache, fatigue, nausea, vomiting, nasal congestion, chest pain or pressure, confusion, low oxygen, and others. In some patients, COVID-19 can cause pneumonia and/or acute respiratory distress syndrome (ARDS). ARDS can be the result of cytokine storm syndrome. In some patients, even those with relatively mild disease symptoms can last for more than a month, or even more than two, three, four, five, six, seven, eight, nine, ten, eleven, or twelve months. COVID-19 severity and progression can be measured using the World Health Organization’s eight point ordinal scale. World Health Organization. (2020). WHO R&D Blueprint: novel Coronavirus: COVID-19 Therapeutic Trial Synopsis, Feb. 18, 2020, Geneva, Switzerland. World Health Organization.

Types of parainfluenza include but are not limited to Human parainfluenza virus type 1 (HPIV-1), type 2 (HPIV-2), type 3 (HPIV-3), type 4 (HPIV-4) and could include other types of parainfluenza that do not yet exist or that have not been described or discovered.

Hantaviruses belong to the bunyavirus family of viruses. There are 5 genera within the family: bunyavirus, phlebovirus, nairovirus, tospovirus, and hantavirus. Each is made up of negative-sensed, single-stranded RNA viruses. All these genera include arthropod-borne viruses, with the exception of hantavirus, which is rodent-borne. Hantaviruses can also infect humans. Hantaviruses could also include other types of parainfluenza that do not yet exist or that have not been described or discovered.

Types of cytomegalovirus include but are not limited to Aotine betaherpesvirus, Cebine betaherpesvirus, Cercopithecine betaherpesvirus, Human betaherpesvirus, Macacine betaherpesvirus, Macacine betaherpesvirus, Mandrilline betaherpesvirus, Panine betaherpesvirus, Papiine betaherpesvirus, Papiine betaherpesvirus, Saimiriine betaherpesvirus, and could include other types of parainfluenza that do not yet exist or that have not been described or discovered.

Herpes viruses include but are not limited to HHV-1(Herpes simplex virus-1 (HSV-1)), HHV-2 (Herpes simplex virus-2 (HSV-2)), HHV-3 (Varicella zoster virus (VZV)), HHV-4 (Epstein-Barr virus (EBV) Lymphocryptovirus), HHV-5 (Cytomegalovirus (CMV)), HHV-6A and 6B (Roseolovirus) HHV-7, HHV-8 (Kaposi’s sarcoma-associated herpesvirus (KSHV)), CeHV-1 (Cercopithecine herpesvirus 1, (monkey B virus)), MuHV-4 (Murid herpesvirus 68 (MHV-68)). There are many other types of known herpes viruses, and herpes viruses could also include other types of parainfluenza that do not yet exist or that have not been described or discovered.

Orthopoxviruses include but are not limited to smallpox, cowpox, horsepox, camelpox, and monkeypox. Orthpoxviruses could also include other types of parainfluenza that do not yet exist or that have not been described or discovered.

The methods of the invention can be used to treat cytokine storm syndrome, regardless of what causes or caused the cytokine storm. For example, a cytokine storm can be caused by respiratory viral, bacterial, parasitic or fungal infection, acute respiratory distress syndrome, trauma, reaction to a blood transfusion, reaction to a medication, graft vs. host disease, gastric aspiration, sepsis, hemophagocytic lymphohistiocytosis, other causes, and combinations thereof. Types of bacteria contemplated by the invention include but are not limited to Staphylococcus species, Streptococcus species, Pseudomonas aeruginosa, Bacteroides species, Clostridium perfringens, Enterococus species, Klebsiella species, Escherichia coli, Pneumococcus species, Listeria monocytogenes, Fusobacterium, Peptococcus, Peptostreptococcus, Enterobacter, Hemophilus influzenae, Proteus, Leptospira, Burkholderia mallei, Burkholderia pseudomallei, Brucellosis, Coxiella burnetti, Vibrio species, Francisella tularensis, Neisseria meningitides, Mycoplasma pneumoniae, Mycobacterium avium, Mycobacterium avium subsp. paratuberculosis, and others.

Types of parasites and protozoans contemplated by the invention include but are not limited to Pneumocystis species, toxoplasmosis, malaria (Plasmodium species), Entamoeba histolytica, Schistosoma mansoni, Trypanosoma species, Leischmania species, and others. Parasites include but are not limited to protozoa (e.g. Sarcodina, Mastigophora, Ciliophora, Sporozoa, and others), helminths (e.g. platyhelminths, acanthocephalins, nematodes, and others), ectoparaisites (e.g. arthropods, and others), and others.

Types of fungus contemplated by the invention include but are not limited to Cryptococcus neoformans, Blastomyces dermatitidis, Histoplasma capsulatum, Candida species, Aspergillus, Coccidioides immitis, Paracoccidioides brasiliensis, Sporothrix schenckii, Rhizopus, Rhizomucor, Mucor, Absidia (Licthemia), and others.

In some cases, pathogenic microorganisms cannot be easily eradicated by the use of existing antimicrobial drugs as these microorganisms may acquire resistance to drugs, or the drugs may pose undesirable side effects to varying degrees to patients. As a result, known antibiotics, antivirals, antifungals, and antiparasitics have not been entirely satisfactory in terms of their activity, behavior in the body, safety, or ability to suppress drug-resistant organisms.

The present invention contemplates the treatment and/or prevention of infections by drug resistant organisms, especially those cause bacteria, fungi, viruses, and parasites. Drug resistant organisms can be resistant to one or more drugs. For example, multi-drug resistant organisms can be multi-drug resistant bacteria, multi-drug resistant viruses, multi-drug resistant fungi, multi-drug resistant parasites, and/or other multi-drug resistant organisms.

In some embodiments, the bacterium is selected from the group consisting of Acinetobacter baumanii, Burkholderia cepacia, Bacterioides fragilis, Chlamydia trachomatis, Citrobacter freundii, Campylobacter jejuni, Escherichia coli, Enterobacter aerogenes, Enterobacter cloacae, Haemophilusinfb, Helicobacter pylori, Klebsiella oxytoca, K. pneumonia (MDR/CRE), Legionella pneumophila, Neisseria meningitides, Neisseria gonorrhoeae, Pseudomonas aeruginosa, Salmonella typhi, paratyphi, typhimurium, Serratia marcescens, Shigella flexneri, Stenotrophomonas maltophilia, Yersinia pseudotuberculosis, Bacillus subtilis, Clostridium neoformans, C. difficile, C. perfringens, Corynebacterium spp, Enterococcus faecalis, Enterococcus faecium, vancomycin-resistant Enterococci (VRE), Listeria monocytogenes, Mycobactrium avium, M. tuberculosis, M. leprae, Nocardia farcinica, P. acnes, Staphylococcus aureus, methicillin-susceptible Staphylococcus aureus (MSSA), methicillin-resistant Staphylococcus aureus (MRSA), Staphylococcus epidermidis, Streptococcus pyogenes, Strep Group A, Strep Group B (agalactiae) and Strep Group C.

In some embodiments, the bacterium is an antibiotic-resistant bacterium. In some embodiments, the bacterium is a multi-drug resistant bacterium. In certain embodiments, the antibiotic-resistant bacterium or the multi-drug resistant bacterium is selected from the group consisting of Acinetobacter baumanii, Escherichia coli, Klebsiella oxytoca, K. pneumonia (MDR/CRE), Pseudomonas aeruginosa, C. difficile, vancomycin-resistant Enterococci (VRE) and methicillin-resistant Staphylococcus aureus (MRSA).

In some embodiments, the fungus is selected from the group consisting of Aspergillus spp, Blastomyces, Candida albicans, glabrata, guilliermondii, krusei, parapsilosis, tropicalis Cryptococcus, Fusarium spp., Mucor spp., Saccharomyces, and Pneumocystis jirovecii (carinii). In some embodiments, the virus is selected from the group consisting of Dengue virus, Ebola virus, EBV, Hepitis A virus, Hepitis B virus, Hepitis C virus, Hepitis D virus, HIV, HSV 1, HSV 2, Cytomegalovirus (CMV), Influenza A virus, Marburg virus, Human respiratory syncytial virus (RSV), SARS-CoV, West Nile virus, Human papillomavirus (HPV), Human rhinoviruses (HRVs), and Zica virus.

In some embodiments, the parasite is selected from the group consisting of Cryptosporidium, Leishmania, Malaria, Schistosoma, Trichomonasm and Trypanosoma.

In some embodiments, the pathogen is a mycoplasma. In certain embodiments, the mycoplasma is selected from the group consisting of M. pneumoniae, M. hominis and M. orale.

The present invention also contemplates the treatment of autoimmune disease. Autoimmune diseases are characterizing by the immune system of a patient recognizing the patient’s own body as foreign, causing the patient’s own immune system to inappropriately attacking the patient’s own body. The present invention now recognizes that modulation of a patient’s immune system, for example by blood irradiation can be used to treat certain aspects of autoimmune disease. Modulation of the immune system can be accomplished by modulating immune, bone marrow, and/or blood cells. Modulation of the immune system can also involve autologous and/or allogeneic bone marrow transplantation, including bone marrow transplantation with or without myeloablation.

Autoimmune diseases include but are not limited to Crohn’s disease, eczema, psoriasis, psoriatic arthritis, rheumatoid arthritis, systemic lupus erythematosus, sarcoidosis, granulomatosis with polyangiitis, myocarditis, inflammatory bowel disease, multiple sclerosis, type 1 diabetes mellitus, Guillain-Barre syndrome, chronic inflammatory demyelinating polyneuropathy, multiple sclerosis Graves’s disease, and myasthenia graves. Further autoimmune diseases are listed in Table 1.

TABLE 1

List of autoimmune diseases

| | | | |
|---|---|---|---|
| Achalasia | Devic’s disease (neuromyelitis optica) | Lyme disease chronic | Pyoderma gangrenosum |
| Addison’s disease | Discoid lupus | Meniere’s disease | Raynaud’s phenomenon |
| Adult Still’s disease | Dressler’s syndrome | Microscopic polyangiitis (MPA) | Reactive Arthritis |
| Agammaglobulinemia | Endometriosis | Mixed connective tissue disease (MCTD) | Reflex sympathetic dystrophy |
| Alopecia areata | Eosinophilic esophagitis (EoE) | Mooren’s ulcer | Relapsing polychondritis |
| Amyloidosis | Eosinophilic fasciitis | Mucha-Habermann disease | Restless legs syndrome (RLS) |
| Ankylosing spondylitis | Erythema nodosum | Multifocal Motor Neuropathy (MMN) or MMNCB | Retroperitoneal fibrosis |
| Anti-GBM/Anti-TBM nephritis | Essential mixed cryoglobulinemia | Multiple sclerosis | Rheumatic fever |
| Antiphospholipid syndrome | Evans syndrome | Myasthenia gravis | Rheumatoid arthritis |
| Autoimmune angioedema | Fibromyalgia | Myositis | Sarcoidosis |
| Autoimmune dysautonomia | Fibrosing alveolitis | Narcolepsy | Schmidt syndrome |
| Autoimmune encephalomyelitis | Giant cell arteritis (temporal arteritis) | Neonatal Lupus | Scleritis |
| Autoimmune hepatitis | Giant cell myocarditis | Neuromyelitis optica | Scleroderma |
| Autoimmune inner ear disease (AIED) | Glomerulonephritis | Neutropenia | Sjögren’s syndrome |
| Autoimmune myocarditis | Goodpasture’s syndrome | Ocular cicatricial pemphigoid | Sperm & testicular autoimmunity |
| Autoimmune oophoritis | Granulomatosis with Polyangiitis | Optic neuritis | Stiff person syndrome (SPS) |
| Autoimmune orchitis | Graves’ disease | Palindromic rheumatism (PR) | Subacute bacterial endocarditis (SBE) |
| Autoimmune pancreatitis | Guillain-Barre syndrome | PANDAS | Susac’s syndrome |
| Autoimmune retinopathy | Hashimoto’s thyroiditis | Paraneoplastic cerebellar degeneration (PCD) | Sympathetic ophthalmia (SO) |
| Autoimmune urticaria | Hemolytic anemia | Paroxysmal nocturnal hemoglobinuria (PNH) | Takayasu’s arteritis |
| Axonal & neuronal neuropathy (AMAN) | Henoch-Schonlein purpura (HSP) | Parry Romberg syndrome | Temporal arteritis/Giant cell arteritis |
| Baló disease | Herpes gestationis or pemphigoid gestationis (PG) | Pars planitis (peripheral uveitis) | Thrombocytopenic purpura (TTP) |
| Behcet’s disease | Hidradenitis Suppurativa (HS) (Acne Inversa) | Parsonage-Turner syndrome | Tolosa-Hunt syndrome (THS) |
| Benign mucosal pemphigoid | Hypogammalglobulinemia | Pemphigus | Transverse myelitis |
| Bullous pemphigoid | IgA Nephropathy | Peripheral neuropathy | Type 1 diabetes |
| Castleman disease (CD) | IgG4-related sclerosing disease | Perivenous encephalomyelitis | Ulcerative colitis (UC) |
| Celiac disease | Immune thrombocytopenic purpura (ITP) | Pernicious anemia (PA) | Undifferentiated connective tissue disease (UCTD) |
| Chagas disease | Inclusion body myositis (IBM) | POEMS syndrome | Uveitis |
| Chronic inflammatory demyelinating polyneuropathy (CIDP) | Interstitial cystitis (IC) | Polyarteritis nodosa | Vasculitis |
| Chronic recurrent multifocal osteomyelitis (CRMO) | Juvenile arthritis | Polyglandular syndromes type I, II, III | Vitiligo |
| Churg-Strauss Syndrome (CSS) or Eosinophilic Granulomatosis (EGPA) | Juvenile diabetes (Type 1 diabetes) | Polymyalgia rheumatica | Vogt-Koyanagi-Harada Disease |
| Cicatricial pemphigoid | Juvenile myositis (JM) | Polymyositis | |
| Cogan’s syndrome | Kawasaki disease | Postmyocardial infarction syndrome | |
| Cold agglutinin disease | Lambert-Eaton syndrome | Postpericardiotomy syndrome | |
| Congenital heart block | Leukocytoclastic vasculitis | Primary biliary cirrhosis | |
| Coxsackie myocarditis | Lichen planus | Primary sclerosing cholangitis | |
| CREST syndrome | Lichen sclerosus | Progesterone dermatitis | |
| Crohn’s disease | Ligneous conjunctivitis | Psoriasis | |
| Dermatitis herpetiformis | Linear IgA disease (LAD) | Psoriatic arthritis | |
| Dermatomyositis | Lupus | Pure red cell aplasia (PRCA) | |

Blood cells can be in whole blood or in a sub-population of cells in whole blood. Subpopulations of cells in whole blood include platelets, red blood cells (erythrocytes), platelets and white blood cells (i.e., peripheral blood leukocytes, which are made up of neutrophils, lymphocytes, eosinophils, basophils and monocytes). These five types of white blood cells can be further divided into two groups, granulocytes (which are also known as polymorphonuclear leukocytes and include neutrophils, eosinophils and basophils) and mononuclear leukocytes (which include monocytes and lymphocytes). Lymphocytes can be further divided into T cells, B cells and NK cells. Peripheral blood cells are found in the circulating pool of blood and not sequestered within the lymphatic system, spleen, liver, or bone marrow. Other cells are present in blood that can be isolated. Types of leukocytes include naive B cells, memory B cells, plasma cells, CD8 T cells, naive CD4 T cells, CD4 memory RO unactivated T cells, CD4 memory RO activated T cells, follicular helper T cells, regulatory T cells, gamma delta T cells, unstimulated NK cells, stimulated NK cells, monocytes, macrophages M0, macrophages M1, macrophages M2, unstimulated dendritic cells, stimulated dendritic cells, unstimulated mast cells, stimulated mast cells, eosinophils, and neutrophils.

Blood can be fractionated to yield various preparations of blood fractions. Blood fractions include but are not limited to red blood cells, white blood cells, platelets, and plasma. Other blood fractions include but are not limited to albumin, clotting factors, colony stimulating factors, cryoprecipitate, erythropoietin (EPO), Factor I, Factor II, fibrinogen/fibrin, hemoglobin-based oxygen carriers, immunoglobulins, interferon, interleukin, prothrombin, Rh Factor, sealants, and thrombin. A blood fraction can also be a fraction that includes an infectious agent, for example a fraction enriched with, relative to whole blood, bacteria, virus, fungus, parasite, or combinations thereof. A fraction can also contain completely or incompletely purified or isolated bacteria, virus, fungus, parasite, or combinations thereof. The present invention contemplates irradiation of whole blood and/or one or more blood fractions.

Herein, “portion” refers to an amount of whole blood or to a fraction of whole blood. The invention contemplates irradiation of a whole portion of blood, a percentage or amount of a portion of blood, a fraction of a portion of blood, a component from a portion of blood, or combinations thereof. The invention contemplates placing into a patient a whole portion of blood, a percentage or amount of a portion of blood, a fraction of a portion of blood, a component from blood, or combinations thereof.

The invention contemplates the removal of a portion of blood from a patient and/or donor and irradiation of the portion of blood, which includes a/the whole portion of blood, a/the percentage or amount of a portion of blood, a/the fraction of a portion of blood, a/the component from a portion blood, or combinations thereof. A fraction and/or a component, for example an infectious agent, can be removed from the portion of blood, irradiated, and placed into a/the patient. After removal from blood, for example a/the portion of blood, an infectious agent can be cultured, expanded, or otherwise increased or decreased in quantity, size, health, infectivity, contagiousness, reproductive capacity, virulence, lethality, purity, and/or other measures of infectious agent characteristics prior to being irradiated and then placed into a/the patient. For example, an infectious agent (e.g. a bacterium, virus, fungus, and/or parasite) can be isolated from a portion of blood and cultured to increase in number, and then the agent can be irradiated and placed into a/the patient. Alternatively, an agent can be irradiated prior to being taken from the portion of blood and placed into a/the patient. The whole infectious agent or one or more pieces of the agent can be placed into a/the patient. For example, an agent can be irradiated, and then antigens from the irradiated agent can be enriched in purity and then placed into a/the patient. These procedures are expected to induce an immune response in a/the patient, as in the vaccine-like effect described herein, and/or to have other effects.

Vaccines are used to protect a patient from a disease, oftentimes an infectious and/or microbial disease or cancer. Vaccines induce a protective immunological response in a patient such that resistance to new infection will be enhanced and/or the clinical severity of the disease reduced. Vaccines typically work by presenting antigens from a target, for example a target organism or cancer cell, to a patient’s immune system. The patient’s immune system recognizes the antigen(s) and mounts a protective response caused by the antigen(s) that confers resistance to the target. The present infection contemplates the use of blood irradiation to induce a vaccine-like effect in a patient.

The vaccine-like effect of the present invention involves the use of radiation to create antigens in the blood of a patient, against which antigens the patient’s immune system can mount an immunological response. For example, the blood of a patient can be irradiated so as to induce damage to blood components, such as blood cells, or other materials in the blood, for example viruses or bacteria. Irradiation can induce damage in a potential target to cause the exposure and/or formation of antigens that can be used by the patient’s immune system to stimulate and mount a response to protect against the target. Irradiation can also induce the exposure and/or formation, of antigens through means other than damage.

Ultraviolet blood irradiation (UBI)—originally the Knott technique—has been used in the United States since 1928 for the successful extracorporeal treatment of microbial infections. Over the years there have been scientific arguments concerning the mechanism by which UBI works and the consensus appears to be that some organisms are killed and a stimulated immune system then protects the patient by clearing the remaining organisms from the body.

Ultraviolet (UV) blood irradiation (UBI) therapy may be administered by a device called a Knott HEMO-IRRADIATOR®, for example as disclosed in Knott’s U.S. Pat. No. 2,308,516. UBI therapy raises the resistance of the host and is therefore able to control many disease processes. A fundamental effect of UBI is to enhance the biochemical and physiological defenses of the body by the introduction of UV energy into the blood stream.

Such treatment is intravenously applied by irradiating blood with a controlled amount of UV energy in the accepted therapeutic band. This produces a rapid detoxifying effect with subsidence of toxic symptoms. Venous oxygen is increased in patients with depressed blood oxygen values. Of special interest is that a rapid rise in resistance to acute or chronic viral and bacterial infection occurs. Minimal harmful effects have been observed with UBI therapy in cases of viral infections, hepatitis, bacterial infections, hypoxemia and many other illnesses, especially blood-related infections.

The Knott technique of blood irradiation (approved by the American Blood Irradiation Society) has achieved the following physiologic objectives: (1) increases the blood oxygen level; (2) increases phagocytosis; (3) relieves toxemia; (4) decreases edema; and (5) controls nausea and vomiting.

In some embodiments of the present invention, treatment generally consists of withdrawing from 1.0 to 1.5 cc of blood per pound of body weight from the patient, and, by use of the Knott HEMO-IRRADIATOR®, or similar device, exposing the blood to radiant energy between the wave lengths of 2,000 and 12,000 angstroms units as it passes through the irradiation unit at a predetermined flow rate. The blood is returned to the patient through the needle used for the initial venipuncture. Treatment requires from 30-45 minutes. Outpatients rest fifteen minutes, after which time they may resume whatever activity is permitted.

Various parameters of the above treatment protocol can be adjusted. For example, the volume of blood used can be from 1 to 5 milliliters per kilogram of blood, or 1 to 4, or 1 to 3, or 1 to 2, or 2 to 4, or 2 to 3, or about 1, or about 2, or about 3, or about 4, or about 5 milliliters of blood per kilogram of body weight. Various types of ultraviolet light are suitable for UBI procedures of the present invention. For example, the types of ultraviolet light contemplated by the invention include those in Table 2.

TABLE 2

Types of ultraviolet light

| Name | Abbreviation | Wavelength (nm) | Photon energy (eV) |
|---|---|---|---|
| Ultraviolet A | UVA | 315-400 | 3.10-3.94 |
| Ultraviolet B | UVB | 280-315 | 3.94-4.43 |
| Ultraviolet C | UVC | 100-280 | 4.43-12.4 |
| Near ultraviolet | NUV | 300-400 | 3.10-4.13 |
| Middle ultraviolet | MUV | 200-300 | 4.13-6.20 |
| Far ultraviolet | FUV | 122-200 | 6.20-10.16 |
| Hydrogen | H Lyman-α | 121-122 | 10.16-10.25 |
| Vacuum ultraviolet | VUV | 10-200 | 6.20-124 |
| Extreme ultraviolet | EUV | 10-121 | 10.25-124 |

UBI is useful to treat various aspects of viral infection, for example to reduce viral load or reduce the risk and/or severity of cytokine storm syndrome. Cytokine storm syndrome is also known as a cytokine storm or cytokine storm syndrome. Cytokine storm syndrome is a dangerous condition caused by some types of virus, for example influenza (e.g. H5N1 influenza and H1N1 influenza) and coronavirus (e.g. SARS-CoV-2, SARS-CoV, and MERS-CoV). Cytokine storm syndrome is a systemic inflammatory response syndrome, so called because it involves massive release of cytokines, that can lead to serious injury to a patient or death. In some embodiments, cytokine storm syndrome is determined by the following three criteria: 1) elevated circulating cytokine levels, 2) acute systemic inflammatory symptoms, and 3) either secondary organ dysfunction (often renal, hepatic, or pulmonary) due to inflammation beyond that which could be attributed to a normal response to a pathogen (if a pathogen is present), or any cytokine-driven organ dysfunction (if no pathogen is present). Examples of cytokines are provided in Table 3.

During a cytokine storm, inflammatory mediators, for example pro-inflammatory cytokines such as Interleukin-1 (IL1), Interleukin-6 (IL6), tumor necrosis factor-alpha (TNF-alpha), oxygen free radicals, and coagulation factors are released by the immune cells of the body. Cytokine storm severity and progression can be measured by levels of cytokines, for example those in Table 3.

TABLE 3

List of cytokines

| | | | | | | | | |
|---|---|---|---|---|---|---|---|---|
| UniProt Entry | UniProt Entry name | Gene names | UniProt Entry | UniProt Entry name | Gene names | UniProt Entry | UniProt Entry name | Gene names |
| Q16663 | CCL15_HUMAN | CCL15 MIP5 NCC3 SCYA15 | P05013 | IFNA6_HUMAN | IFNA6 | P32970 | CD70_HUMAN | CD70 CD27L CD27LG TNFSF7 |
| P13501 | CCL5_HUMAN | CCL5 D17S136E SCYA5 | P32881 | IFNA8_HUMAN | IFNA8 | P34820 | BMP8B_HUMAN | BMP8B BMP8 |
| P04141 | CSF2_HUMAN | CSF2 GMCSF | Q8IU54 | IFNL1_HUMAN | IFNL1 IL29 ZCYTO21 | Q9UBH0 | I36RA_HUMAN | IL36RN FIL1D IL1F5 IL1HY1 IL1L1 IL1RP3 UNQ1896/ PRO4342 |
| P09919 | CSF3_HUMAN | CSF3 C17orf33 GCSF | Q9Y4X3 | CCL27_HUMAN | CCL27 ILC SCYA27 | P13232 | IL7_HUMAN | IL7 |
| Q96DZ9 | CKLF5_HUMAN | CMTM5 CKLFSF5 | O00626 | CCL22_HUMAN | CCL22 MDC SCYA22 A-152E5.1 | P15248 | IL9_HUMAN | IL9 |
| P16619 | CL3L1_HUMAN | CCL3L1 D17S1718 G0S19-2 SCYA3L1; CCL3L3 | Q8IZV2 | CKLF8_HUMAN | CMTM8 CKLFSF8 | Q8IZJ0 | IFNL2_HUMAN | IFNL2 IL28A ZCYTO20 |
| Q99731 | CCL19_HUMAN | CCL19 ELC MIP3B SCYA19 | Q96FZ5 | CKLF7_HUMAN | CMTM7 CKLFSF7 | P01566 | IFN10_HUMAN | IFNA10 |
| P13500 | CCL2_HUMAN | CCL2 MCP1 SCYA2 | O60565 | GREM1_HUMAN | GREM1 CKTSF1B1 DAND2 DRM PIG2 | P01579 | IFNG_HUMAN | IFNG |
| Q16627 | CCL14_HUMAN | CCL14 NCC2 | O14793 | GDF8_HUMAN | MSTN GDF8 | Q8TAD2 | IL17D_HUMAN | IL17D UNQ3096/ PRO21175 |
| Q9UBD9 | CLCF1_HUMAN | CLCF1 BSF3 CLC NNT1 | O43927 | CXL13_HUMAN | CXCL13 BCA1 BLC SCYB13 | Q9UHD0 | IL19_HUMAN | IL19 ZMDA1 |
| P22003 | BMP5_HUMAN | BMP5 | Q9NPF7 | IL23A_HUMAN | IL23A SGRF UNQ2498/ PRO5798 | Q9P0M4 | IL17C_HUMAN | IL17C UNQ561/P RO1122 |
| Q7Z5Y6 | BMP8A_HUMAN | BMP8A | P02775 | CXCL7_HUMAN | PPBP CTAP3 CXCL7 SCYB7 TGB1 THBGB1 | Q9UHF5 | IL17B_HUMAN | IL17B IL20 NIRF ZCYTO7 UNQ516/P RO1031 |
| O95813 | CER1_HUMAN | CER1 DAND4 | Q07325 | CXCL9_HUMAN | CXCL9 CMK MIG SCYB9 | P015 83 | IL1A_HUMAN | IL1A IL1F1 |
| P49771 | FLT3L_HUMAN | FLT3LG | Q9H293 | IL25_HUMAN | IL25 IL17E UNQ3120/ PRO 10272 | Q9NZH7 | IL36B_HUMAN | IL36B IL1F8 IL1H2 |
| P15514 | AREG_HUMAN | AREG AREGB SDGF | Q8NEV9 | IL27A_HUMAN | IL27 IL27A IL30 | O00175 | CCL24_HUMAN | CCL24 MPIF2 SCYA24 |
| Q99616 | CCL13_HUMAN | CCL13 MCP4 NCC1 SCYA13 | Q6EBC2 | IL31_HUMAN | IL31 | P29459 | IL12A_HUMAN | IL12A NKSF1 |
| Q9NRJ3 | CCL28_HUMAN | CCL28 SCYA28 | O95760 | IL33_HUMAN | IL33 C9orf26 IL1F11 NFHEV | P14174 | MIF_HUMAN | MIF GLIF MMIF |
| 015444 | CCL25_HUMAN | CCL25 SCYA25 TECK | Q9UHA7 | IL36A_HUMAN | IL36A FIL1E IL1E IL1F6 | Q86WN2 | IFNE_HUMAN | IFNE IFNE1 UNQ360/P RO655 |
| P27539 | GDF1_HUMAN | GDF1 | Q8NHW4 | CC4L_HUMAN | CCL4L1 CCL4L LAG1 SCYA4L1; CCL4L2 CCL4L SCYA4L2 | P20809 | IL11_HUMAN | IL11 |
| P28799 | GRN_HUMAN | GRN | P12643 | BMP2_HUMAN | BMP2 BMP2A | Q1L6U9 | MSMP_HUMAN | MSMP PSMP |
| O95390 | GDF11_HUMAN | GDF11 BMP 11 | O95393 | BMP10_HUMAN | BMP 10 | P01562 | IFNA1_HUMAN | IFNA1; IFNA13 |
| P41273 | TNFL9_HUMAN | TNFSF9 | O00585 | CCL21_HUMAN | CCL21 SCYA21 UNQ784/P RO1600 | P01568 | IFN21_HUMAN | IFNA21 |
| P13236 | CCL4_HUMAN | CCL4 LAG1 MIP1B SCYA4 | P19876 | CXCL3_HUMAN | CXCL3 GRO3 GROG SCYB3 | K9M1U5 | IFNL4_HUMAN | IFNL4 |
| Q9UBR5 | CKLF_HUMAN | CKLF CKLF1 HSPC224 UNQ410/P RO772 | P42830 | CXCL5_HUMAN | CXCL5 ENA78 SCYB5 | P05231 | IL6_HUMAN | IL6 IFNB2 |
| P22362 | CCL1_HUMAN | CCL1 SCYA1 | O95715 | CXL14_HUMAN | CXCL14 MIP2G NJAC SCYB14 PSEC0212 UNQ240/P RO273 | Q9NZH8 | IL36G_HUMAN | IL36G IL1E IL1F9 IL1H1 IL1RP2 UNQ2456/ PRO5737 |
| Q9BXJ3 | C1QT4_HUMAN | C1QTNF4 CTRP4 | O75610 | LFTY1_HUMAN | LEFTY 1 LEFTB LEFTYB UNQ278/P RO317 | P10147 | CCL3_HUMAN | CCL3 G0S19-1 MIP1A SCYA3 |
| Q96MX0 | CKLF3_HUMAN | CMTM3 CKLFSF3 | P55107 | GDF10_HUMAN | GDF10 BMP3B | Q92583 | CCL17_HUMAN | CCL17 SCYA17 TARC |
| Q8IZ96 | CKLF1_HUMAN | CMTM1 CKLFSF1 | Q6KF10 | GDF6_HUMAN | GDF6 BMP 13 GDF16 | P51671 | CCL11_HUMAN | CCL11 SCYA11 |
| P22004 | BMP6_HUMAN | BMP6 VGR | P40933 | IL15_HUMAN | IL15 | Q14116 | IL18_HUMAN | IL18 IGIF IL1F4 |
| Q9Y258 | CCL26_HUMAN | CCL26 SCYA26 UNQ216/P RO242 | Q14213 | IL27B_HUMAN | EBI3 IL27B | Q96QR1 | SG3A1_HUMAN | SCGB3A1 HIN1 PNSP2 UGRP2 UNQ629/P RO1245 |
| Q8TAZ6 | CKLF2_HUMAN | CMTM2 CKLFSF2 | P05113 | IL5_HUMAN | IL5 | O43557 | TNF14_HUMAN | TNFSF14 HVEML LIGHT UNQ391/P RO726 |
| P43026 | GDF5_HUMAN | GDF5 BMP14 CDMP1 | P10145 | IL8_HUMAN | CXCL8 IL8 | P48061 | SDF1_HUMAN | CXCL12 SDF1 SDF1A SDF1B |
| 015467 | CCL16_HUMAN | CCL16 ILINCK NCC4 SCYA16 | P05112 | IL4_HUMAN | IL4 | P32971 | TNFL8_HUMAN | TNFSF8 CD30L CD30LG |
| Q99988 | GDF15_HUMAN | GDF15 MIC1 PDF PLAB PTGFB | P01574 | IFNB_HUMAN | IFNB 1 IFB IFNB | O43508 | TNF12_HUMAN | TNFSF12 APO3L DR3LG UNQ181/P RO207 |
| P58499 | FAM3B_HUMAN | FAM3B C21orf11 C21orf76 PRED44 UNQ320/P RO365 | P29460 | IL12B_HUMAN | IL12B NKSF2 | Q9Y275 | TN13B_HUMAN | TNFSF13B BAFF BLYS TALL1 TNFSF20 ZTNF4 UNQ401/P RO738 |
| P12645 | BMP3_HUMAN | BMP3 BMP3A | P08700 | IL3_HUMAN | IL3 | P80098 | CCL7_HUMAN | CCL7 MCP3 SCYA6 SCYA7 |
| P13497 | BMP1_HUMAN | BMP1 PCOLC | Q9UK05 | GDF2_HUMAN | GDF2 BMP9 | O75888 | TNF13_HUMAN | TNFSF13 APRIL TALL2 ZTNF2 UNQ383/P RO715 |
| P80162 | CXCL6_HUMAN | CXCL6 GCP2 SCYB6 | P09341 | GROA_HUMAN | CXCL1 GRO GRO1 GROA MGSA SCYB1 | P40225 | TPO_HUMAN | THPO MGDF |
| Q13007 | IL24_HUMAN | IL24 MDA7 ST16 | O60383 | GDF9_HUMAN | GDF9 | Q969D9 | TSLP_HUMAN | TSLP |
| Q9NPH9 | IL26_HUMAN | IL26 AK155 | Q7Z4P5 | GDF7_HUMAN | GDF7 | P43490 | NAMPT_HUMAN | NAMPT PBEF PBEF1 |
| P19875 | CXCL2_HUMAN | CXCL2 GRO2 GROB MIP2A SCYB2 | Q16552 | IL17_HUMAN | IL17A CTLA8 IL17 | Q86YJ6 | THNS2_HUMAN | THNSL2 |
| Q8WWZ1 | IL1FA_HUMAN | IL1F10 FIL1T IL1HY2 IL38 FKSG75 UNQ6119/ PRO20041 | P29965 | CD40L_HUMAN | CD40LG CD40L TNFSF5 TRAP | P01374 | TNFB_HUMAN | LTA TNFB TNFSF1 |
| P02778 | CXL10_HUMAN | CXCL10 INP10 SCYB10 | P05015 | IFN16_HUMAN | IFNA16 | P48023 | TNFL6_HUMAN | FASLG APT1LG1 CD95L FASL TNFSF6 |
| 014625 | CXL11_HUMAN | CXCL11 ITAC SCYB 11 SCYB9B | P05014 | IFNA4_HUMAN | IFNA4 | Q6UWK7 | GP15L_HUMAN | GPR15L C10orf99 UNQ1833/ PRO3446 |
| Q9H2A7 | CXL16_HUMAN | CXCL16 SCYB16 SRPSOX UNQ2759/ PRO6714 | Q16619 | CTF1_HUMAN | CTF1 | P02776 | PLF4_HUMAN | PF4 CXCL4 SCYB4 |
| Q9NYY1 | IL20_HUMAN | IL20 ZCYTO10 UNQ852/P RO1801 | O95972 | BMP15_HUMAN | BMP15 GDF9B | Q96PD4 | IL17F_HUMAN | IL17F |
| Q9HBE4 | IL21_HUMAN | IL21 | P06744 | G6PI_HUMAN | GPI | P55000 | SLUR1_HUMAN | SLURP 1 ARS |
| Q9GZX6 | IL22_HUMAN | IL22 ILTIF ZCYTO18 UNQ3099/ PRO 10096 | Q96S42 | NODAL_HUMAN | NODAL | Q7Z5A7 | TAFA5_HUMAN | TAFA5 FAM19A5 UNQ5208/ PRO34524 |
| P60568 | IL2_HUMAN | IL2 | P78556 | CCL20_HUMAN | CCL20 LARC MIP3A SCYA20 | P50591 | TNF10_HUMAN | TNFSF10 APO2L TRAIL |
| P24001 | IL32_HUMAN | IL32 NK4 TAIF | Q8IZ19 | IFNL3_HUMAN | IFNL3 IL28B IL28C ZCYTO22 | Q9UNG2 | TNF18_HUMAN | TNFSF18 AITRL GITRL TL6 UNQ149/P RO175 |
| Q6ZMJ4 | IL34_HUMAN | IL34 C16orf77 | P80075 | CCL8_HUMAN | CCL8 MCP2 SCYA10 SCYA8 | O95150 | TNF15_HUMAN | TNFSF15 TL1 VEGI |
| Q9P0W0 | IFNK_HUMAN | IFNK UNQ6124/ PRO20084 | Q9NR23 | GDF3_HUMAN | GDF3 UNQ222/P RO248 | P78423 | X3CL1_HUMAN | CX3CL1 FKN NTT SCYD1 A-152E5.2 |
| Q9H772 | GREM2_HUMAN | GREM2 CKTSF1B2 DAND3 PRDC | P12644 | BMP4_HUMAN | BMP4 BMP2B DVR4 | P01375 | TNFA_HUMAN | TNF TNFA TNFSF2 |
| P01584 | IL1B_HUMAN | IL1B IL1F2 | P01567 | IFNA7_HUMAN | IFNA7 | Q06643 | TNFC_HUMAN | LTB TNFC TNFSF3 |
| O00292 | LFTY2_HUMAN | LEFTY2 EBAF LEFTA LEFTYA TGFB4 PSEC0024 | Q14005 | IL16_HUMAN | IL16 | 014788 | TNF11_HUMAN | TNFSF11 OPGL RANKL TRANCE |
| P01569 | IFNA5_HUMAN | IFNA5 | Q6UX52 | IL40_HUMAN | C17orf99 IL40 UNQ464/P RO809 | P23510 | TNFL4_HUMAN | TNFSF4 TXGP1 |
| Q9NZH6 | IL37_HUMAN | IL37 FIL1Z IL1F7 IL1H4 IL1RP1 | P15018 | LIF_HUMAN | LIF HILDA | Q6UX27 | VSTM1_HUMAN | VSTM1 UNQ3033/ PRO9835 |
| P55773 | CCL23_HUMAN | CCL23 MIP3 MPIF1 SCYA23 | P01571 | IFN17_HUMAN | IFNA17 | Q9UBD3 | XCL2_HUMAN | XCL2 SCYC2 |
| P01570 | IFN14_HUMAN | IFNA14 | P05000 | IFNW1_HUMAN | IFNW1 | P47992 | XCL1_HUMAN | XCL1 LTN SCYC1 |
| P01563 | IFNA2_HUMAN | IFNA2 IFNA2A IFNA2B IFNA2C | P35225 | IL13_HUMAN | IL13 NC30 | Q12904 | AIMP1_HUMAN | AIMP 1 EMAP2 SCYE1 |
| P22301 | IL10_HUMAN | IL10 | P13725 | ONCM_HUMAN | OSM | P09603 | CSF1_HUMAN | CSF1 |
| P18075 | BMP7_HUMAN | BMP7 OP1 | P10451 | OSTP_HUMAN | SPP1 BNSP OPN PSEC0156 | | | |
| P55774 | CCL18_HUMAN | CCL18 AMAC1 DCCK1 MIP4 PARC SCYA18 | P10720 | PF4V_HUMAN | PF4V1 CXCL4V1 SCYB4V1 | | | |

Cytokine storms have the potential to cause significant damage to body tissues and organs. For example, occurrence of cytokine storms in the lungs can cause an accumulation of fluids and immune cells, for example macrophages, in the lungs, and eventually block the body’s airways thereby resulting in respiratory distress and even death.

It is important to be able to prevent, control, or mitigate the occurrence of the cytokine storm. There are existing or conventional techniques associated with preventing, controlling, or mitigating the occurrence of the cytokine storm (and unwanted inflammation in general). For example, it has been reported that TNF inhibitors may be useful for facilitating the control of cytokine storms and for reducing adverse reactions caused by the occurrence of cytokine storms within the body. In addition, research has suggested that angiotensin converting enzyme (ACE) inhibitors, and angiotensin II receptor blockers (ARBs), may have clinical utility for controlling or down-regulating cytokine storms, and for reducing inflammation, within the body. Corticosteriods and non-steroidal anti-inflammatory drugs (NSAIDS) have also been employed in an attempt to treat patients experiencing cytokine storms. In addition, suggested therapeutic agents for treating influenza-type viral infections include antibodies to the influenza virus neuraminidase.

However, the effectiveness and safety of many conventional techniques associated with the prevention, control, or mitigation of cytokine storms within the body have not been comprehensively or adequately verified. In addition, many conventional anti-influenza treatments (e.g. anti-viral or anti-influenza drugs) have associated undesirable side effects when consumed by a patient. Such undesirable side effects include nausea, vomiting, and toxicity. Furthermore, there is an increasing problem of developed resistance to many conventional anti-viral (e.g. anti-influenza) drugs. Accordingly, new or enhanced approaches for at least one of preventing, controlling, or mitigating cytokine storms may be useful for improving public health. Furthermore, new compositions or techniques providing anti-influenza or anti-inflammatory properties may also be useful for improving public health.

In some embodiments of the invention, multiple UBI treatments are administered. For example, UBI treatments can be administered every day, every other day, two times a week, three times a week, four times a week, five times a week, six times a week, about once every ten days, about once every two weeks, about once every three weeks, about once every four weeks, or at other intervals.

Other types of radiation can also be used in the present invention, for example γ radiation. There are many types of radiation. Of particular use for the invention is molecularly damaging radiation. Molecularly damaging radiation can be ionizing radiation or lower energy radiation that is not ionizing but does induce other types of molecular damage. For example, non-ionizing radiation can cause the formation of free radicals that can damage biological molecules. Non-ionizing radiation can also induce the formation of pyrimidine dimers in nucleic acid molecules, and radiation can also heat biological molecules. Near ultraviolet radiation and radiation in the visible spectrum can cause molecular damage, including free radical and pyrimidine dimer formation, and longer wavelength radiation, for example microwave radiation can heat molecules.

Radiation can be particle radiation or electromagnetic radiation. Types of particle radiation include alpha particles, beta particles, protons, helium ions, HZE ions, certain electrons, photons, neutrons, neutrinos, mesons, muons, and others. Electromagnetic radiation consists of electromagnetic waves (or the wave quanta, photons), which can have varied but interrelated wavelengths, frequencies, and energies. Frequency is inversely proportional to wavelength, according to the equation:

$$v = f λ$$

where v is the speed of the wave (c, the speed of light in a vacuum, when in a vacuum or less than c in other media), f is the frequency and λ is the wavelength. A photon has an energy, E, proportional to its frequency, f, by

$$E = h f = h c / λ$$

where h is Planck’s constant, λ is the wavelength and c is the speed of light in a vacuum. Higher energies tend to be more damaging to molecules than lower energies.

High energy radiation has low wavelengths. UV radiation is a type of ionizing radiation, with properties depicted in Table 2, that has lower wavelength and higher energy than radiation in the form of visible light. Ionizing radiation in the form of UV radiation is particularly appropriate for certain embodiments of the invention. γradiation has lower wavelength and higher energy than UV radiation and is a particularly appropriate form of radiation for certain embodiments of the invention. X-ray radiation is another form of low wavelength, high energy electromagnetic radiation that is historically distinguished from γradiation, also electromagnetic radiation. X-rays can be distinguished from γradiation by their source or their wavelengths, frequencies, and energies. X-rays are emitted by electrons outside of the nucleus, while γrays are emitted by the nucleus. However, X-ray radiation and γradiation have overlapping wavelengths, frequencies, and energies and are both appropriate radiation for certain embodiments of the invention. Most X-rays have wavelengths of 10 picometers (pm) to 10 nanometers (nm), and γ rays can be described as radiation with wavelengths less than about 10 pm. Radio, microwave, infrared, and optical radiation are also appropriate for certain embodiments of the invention. The properties of certain types of radiation are depicted in Table 4.

TABLE 4

Wavelengths, frequencies, and energies of types of radiation

| | | | |
|---|---|---|---|
| | Wavelength (m) | Frequency (Hz) | Energy (J) |
| Radio | > 1 × 10⁻¹ | <3 × 10⁹ | < 2 × 10⁻²⁴ |
| Microwave | 1 × 10⁻³ - 1 × 10⁻¹ | 3 × 10⁹ - 3 × 10¹¹ | 2 × 10⁻²⁴- 2 × 10⁻²² |
| Infrared | 7 × 10⁻⁷ - 1 × 10⁻³ | 3 × 10¹¹-4 × 10¹⁴ | 2 × 10⁻²² - 3 × 10⁻¹⁹ |
| Optical | 4 × 10⁻⁷ - 7 × 10⁻⁷ | 4 × 10¹⁴ - 7.5 × 10¹⁴ | 3 × 10⁻¹⁹ - 5 × 10⁻¹⁹ |
| UV | 1 × 10⁻⁸ -4 × 10⁻⁷ | 7.5 × 10¹⁴ - 3 × 10¹⁶ | 5 × 10⁻¹⁹ - 2 × 10⁻¹⁷ |
| X-ray | 1 × 10⁻¹¹ - 1 × 10⁻⁸ | 3 × 10¹⁶ - 3 × 10¹⁹ | 2 × 10⁻¹⁷ - 2 × 10⁻¹⁴ |
| Gamma-ray | < 1 × 10⁻¹¹ | >3 × 10¹⁹ | >2 × 10⁻¹⁴ |

The foregoing methods can be administered in combination with suitable drugs as combination therapies. In each case, the method of treatment of the invention can be administered prior to, at the same time as, or after the administration of a drug for treatment of the condition. In accordance with the methods of the present invention, more than one compound or composition may be co-administered with one or more other compounds, such as known anti-viral compounds or molecules as well as antibiotics, chloroquine, hydroxychloroquine, known drugs for treating pneumonia, an analgesic (such as lidocaine or paracetoamol), an anti-inflammatory (such as bethamethasone, non-steroid anti-inflammatory drugs (NSAIDs), acetaminophen, ibuprofen, naproxen), and/or other suitable drugs.

The foregoing methods can also be administered with further treatment of the portion of blood removed from the patient. For example, the portion of blood can be contacted with a compound selected from riboflavin, psoralens, amotosalen HCl, porphyrins, photosensitizers, chlorophyll derivatives, light-sensitive agents, UV-activated nucleic acid replication inhibitors, dyes (such as such as neutral red, methylene blue, acridine, toluidines, flavine (acriflavine hydrochloride) and phenothiazine derivatives), coumarins, quinolones, quinones, and anthroquinones. The compound and portion of blood can then be irradiated with UV to inactivate pathogens. Other treatments of the portion of blood are also possible.

The foregoing methods can be used to inactivate white blood cells in the portion of blood removed from the patient and/or in the total blood of the patient. Without intending to be bound by theory, exposing blood to UV radiation can have multiple effects. The effects can be killing, weakening, and/or inactivating pathogens, downregulation of the immune system, inactivating, attenuating, and/or downregulating white blood cell activity.

The foregoing methods can be used wherein the patient’s own blood is irradiated and placed back into the patient (autologous) or wherein a donor, who is not the patient, is the source of a portion of blood that is placed into the patient (allogeneic).

### EXAMPLES

### Example 1

A patient with a respiratory viral infection, for example a patient with SARS-CoV-2, is treated with ultraviolet blood irradiation (UBI). The UBI is administered by a variation of the Knott Hemo-irradiator that is compliant with modern safety standards.

First, a portion of blood is removed from the patient. The amount of blood removed is based on the patient’s body weight or blood volume. For example, 3.3 milliliters of blood per kilogram of body weight is removed from the patient. The blood is mixed with heparin to prevent clotting, though under certain circumstances, a person of skill in the art might conclude that the heparin is not necessary or should not be used. For example, a patient with recent hemorrhage might not need heparin due to a risk of bleeding.

The blood is then exposed to ultraviolet light using the irradiator. The irradiator exposes the blood to ultraviolet light with wavelengths between 200 and 400 nanometers. The blood flows through the chamber of the irradiator at a flow rate of 30 milliliter per minute, thereby exposing the blood to the desired amount of ultraviolet radiation. The irradiated blood is then placed back into the patient at the fastest safe infusion rate.

The irradiated blood causes a reduction of the viral load in the patient and/or a reduction in the risk and/or severity of cytokine storm syndrome.

Additional rounds of UBI treatment are conducted under appropriate circumstances.

### Example 2

A patient with a respiratory viral infection, for example a patient with SARS-CoV-2, is treated with ultraviolet blood irradiation (UBI). A portion of blood is removed from the patient and treated with molecularly damaging radiation, such as UV radiation or γradiation. The radiation damages the virus in the portion of blood, causing the exposure of viral antigens. The damage can expose many different types of antigens, including antigens derived from viral proteins, RNA, DNA, modifications to viral components (e.g. posttranslational modifications of viral proteins, posttranscriptional modifications to RNA, and/or others), and other types of antigens. The patient’s immune system recognizes the viral antigens created by the radiation and stimulates an immune response against the virus, causing the patient’s immune system to attack the virus. This is a vaccine-like effect that protects the patient from disease.

### Example 3

A pandemic caused by SARS-CoV-2 has led to an illness termed COVID-19 with significant ongoing morbidity and increasing fatalities (174,336) with a preliminary mortality rate of about 6.9% (Apr. 21, 2020/ https://coronavirus.jhu.edu/map.html). COVID-19 presents with a spectrum of disease from mild and self-limited to severe progressive pneumonia, multiorgan failure, and death. One likely culprit of the high mortality is cytokine storm syndrome (CSS) but no effective therapies exist. In the late 1940s and 1950s ultraviolet blood irradiation (UBI) was safely used to treat over 60,000 patients including those with bacterial and viral infections. With the advent of penicillin in the 1940s and polio vaccine in the 1950s UBI may have been replaced by these and additional antibiotics in subsequent years. This study tests the safety and efficacy of the Mirasol® device (currently used in blood transfusion safety to reduce pathogens in donated blood) to treat hospitalized patients with COVID-19. Using UV light with riboflavin added to whole blood inactivates bacterial, viral and parasitic pathogens, including SARS-CoV-2, also inactivating white blood cells that propagate CSS.

Goals: In hospitalized patients with Covid-19 approximately 450 cc of whole blood will be treated with riboflavin and UV light (Mirasol® device) then retransfusing in an autologous manner, UV blood irradiation (UBI) will cause decreasing cytokine storm, improving illness severity, improve hypoxemia and development of SARS-CoV-2 antibody titers. By acutely inactivating leukocytes, destroying a small proportion of pathogen and shifting the O₂ dissociation curve to the left, improvements over 1, 3 and 7 days in levels of serum biomarkers of CSS, clinical illness severity scores, the PaO₂/FiO₂ ratio will occur and generate antibody titers to SARS-CoV-2 at 14 days.

Implications: UBI with riboflavin for the treatment of viral-associated cytokine storm, along with an improvement in clinical illness severity will be a novel therapeutic approach to combat the harmful effects of SARS-CoV-2 infection and also other infectious and non-infectious illnesses resulting from overwhelming inflammatory responses leading to shock and/or acute respiratory distress syndrome (ARDS) with cytokine storm syndrome (CSS).

Inclusion Criteria:
Provision of signed and dated informed consent form consistent with ICH-GCP
Informed consent can be provided by patient or legally authorized representative
Male or female hospitalized patients age >_ 18 years
Hospitalized for confirmed active Covid-19 infection by PCR or IgM to SARS-CoV-2 in preceding 72 hours; OR PCR positive sample ≥72 hours previous but progressive disease suggestive of ongoing SARS-CoV-2 infection
Lung involvement confirmed by chest imaging
≤14 days since illness onset
Willingness to participate in trial arm they select 1. UBI intervention, blood draws, symptom reporting, 14 d antibodies and 30 day follow up phone call or visit if inpatient status, 2. No UBI intervention, blood draws, symptom reporting, 14 d antibodies and 30 day follow up phone call or visit if inpatient status.

Exclusion Criteria:
Physician makes decision that trial involvement is not in patient’s best interest or any condition that prevents following the trial protocol safely
Inability to have or successful placement of 16 gauge iv placed for blood collection if no central or midline catheter access present
Allergy to riboflavin
Life expectancy < 48 hours
History of pulseless electrical activity in previous 48 hours.
Pregnancy/breast feeding
100% FiO2 requirement when proned
Use of 2 vasopressors at maximum dosage or 3 total vasopressors
Hemoglobin < 10.0 mg/dL
Hyperkalemia with serum potassium currently > 5.0 mEq/L
Active bleeding in previous 48 hours
System fibrinolytic use in previous 48 hours
Requirement for renal replacement therapy initiated and ongoing during current hospitalization (i.e. hemodialysis, CVVH, SLED)
Major injury or surgery needed (e.g. bowel perforation, diverticulitis) at Investigator’s judgment
History of saddle or submassive pulmonary embolus in previous 7 days
History of intracranial hemorrhage or stroke in preceding 7 days or unresolved intracranial hemorrhage
History of advanced hepatic liver disease (Child Pugh C), variceal bleeding, spontaneous bacterial peritonitis, or hepatic encephalopathy
AST, ALT > 5 × upper limit of normal at time of consent
Current history of non-resected metastatic solid tumor on chemotherapy
Anticipated need for sulfa antibiotic, dapsone or sulfasalazine in the 5 days following UBI intervention
History of porphyria
Severe pulmonary hypertension requiring intravenous vasoactive medication
Patients not able to understand or follow trial procedures including completion of self-administered questionnaires

Anticipated transfer or discharge from hospital in next 72 hours.

Outcomes:
Primary-Safety
   1. The cumulative incidence of severe adverse events
Secondary-Efficacy metrics
   1. Time to clinical recovery.
   2. Time to improve by 2 points on clinical severity ordinal 7-point scale.
   3. Changes in clinical illness severity scores (i.e., SOFA, APACHE II, SIC, NEWS2)
   4. Changes in serum biomarkers of inflammation and cytokine storm (i.e. ferritin, hs-CRP, IL-6, LDH, D-dimer)
   5. Changes in oxygenation measured by PaO2/FiO2 ratio
   6. Using a modified flu-iiQ™ validated patient-reported outcomes measure (PROM) to assess clinical improvement.

Study Protocol Timeline:
Stage 1: n=10
   For safety monitoring no more than 1 enrolled per day between 0800-1700 M-F across illness severity spectrum (floor, IMC, ICU).
Stage 2: n= 15
   If no significant adverse safety signals from Phase 1 and Safety Monitoring Committee agrees, proceed with same protocol enrolled between hours of 0800-1700 M-F
Stage 3: n=75
   If no significant adverse safety signals from Phase 2, Safety Monitoring Committee agrees and blood bank/phlebotomy comfortable with the study and Mirasol® device use 7 days/week.

Results: In the current pandemic of Covid-19 triggering CSS and hypoxemia, after treatment with UBI and riboflavin using the MIRASOL® device and ~ 450 cc of a Covid-19 patient’s blood then autologously retransfusing the blood into the same patient, the following results will occur:
1. Use of the Mirasol® device and riboflavin with autologous transfusion of this treated whole blood is safe in hospitalized patients with Covid-19.
2. The time to clinical recovery will improve.
3. Time to improve by 2 points on clinical severity ordinal 7-point scale will improve.
4. The clinical illness severity will decrease in the ensuing 1-30 days measured by clinical illness severity scores (i.e. SOFA, APACHE II, SIC, NEWS2).
5. Via leukocyte inactivation it will decrease the CSS as demonstrated by a reduction in serum biomarkers of CSS at days 1, 3 and 7 following treatment.
6. Via shift of the hemoglobin O2 dissociation curve to the left will lead to an acute improvement in hypoxemia quantified by the PaO2/FiO2 ratio at designated time intervals.
7. Via viral disruption and reintroduction of the inactivated pathogen components it will improve the antibody formation response quantified by antibody titers to SARS-CoV-2 at 14 days.

Variations, modifications, and other implementations of what is described herein will occur to those of ordinary skill without departing from the spirit and the scope of the invention. Accordingly, the invention is not to be limited only to the preceding illustrative description. Each of the embodiments of the invention may be combined individually or in combination with one or more other embodiments of the invention.

Those skilled in the art will recognize or be able to ascertain using no more than routine experimentation, numerous equivalents to the compounds, compositions, and methods of use thereof described herein. Such equivalents are considered to be within the scope of the invention.

The contents of all references, patents and published patent applications cited throughout this Application, as well as their associated figures are hereby incorporated by reference in their entirety.

What is claimed:

1. A method of treating a patient having or being at risk of cytokine storm syndrome comprising:
a. removing a portion of blood from the patient;
b. exposing the portion of blood to ultraviolet radiation;
c. placing the exposed portion of blood into the patient;
wherein the method improves a treatment outcome of the cytokine storm syndrome.

2. A method of treating a patient having or being at risk of cytokine storm syndrome comprising:
a. removing a portion of blood from a donor;
b. exposing the portion of blood to radiation;
c. placing the exposed portion of blood into the patient;
wherein the method improves a treatment outcome of the cytokine storm syndrome.

3. A method of treating a patient having or being at risk of autoimmune disease comprising:
a. removing a portion of blood from a donor;
b. exposing the portion of blood to radiation;
c. placing the exposed portion of blood into the patient;
wherein the method improves a treatment outcome of autoimmune disease.

4. A method of treating a patient having or being at risk of an infectious disease comprising:
a. removing a portion of blood from a donor;
b. exposing the portion of blood to radiation;
c. placing the exposed portion of blood into the patient;
wherein the patient develops an immune response against the infectious disease.

5. A method of modulating leukocytes in a patient:
a. removing a portion of blood from a donor;
b. exposing the portion of blood to radiation;
c. placing the exposed portion of blood into the patient;
wherein the method improves a treatment outcome of a disease.

6. A method of treating a patient having or being at risk of COVID-19 comprising:
a. removing a portion of blood from a donor;
b. exposing the portion of blood to ultraviolet radiation;
c. placing the exposed portion of blood into the patient;
wherein the method improves a treatment outcome of the COVID-19.

7. The method of any one of claims 3-6, wherein the patient has, had, or is at risk of cytokine storm syndrome.

8. The method of any one of claims 1-2 and 7, wherein the cytokine storm syndrome is associated with a condition selected from the group consisting of viral, bacterial, parasitic or fungal infection, acute respiratory distress syndrome, trauma, reaction to a blood transfusion, reaction to a medication, graft vs. host disease, gastric aspiration, sepsis, hemophagocytic lymphohistiocytosis, autoimmune disease, and combinations thereof.

9. The method of claim 8, wherein the condition is a respiratory viral infection by a virus selected from the group consisting of influenza virus, coronavirus, hantavirus, cytomegalovirus, herpesvirus, varicella virus, rhinovirus, parainfluenza virus, human metapneumovirus, adenovirus, respiratory syncytial virus, Ebstein-Barr virus, Dengue virus, West Nile virus, Western and Eastern equine encephalitis viruses, polio, chikungunya variola virus, Ebola virus, and orthopoxvirus.

10. The method of claim 9, wherein the respiratory viral infection is infection by a coronavirus.

11. The method of claim 10, wherein the respiratory viral infection is infection by SARS-CoV-2.

12. The method of any preceding claim, wherein the step of removing comprises removing between about 0.1% and about 20%, between about 1% and about 20%, or between about 5% and about 15% of the blood of the patient.

13. The method of any preceding claim, wherein between about 1.0 and about 5.0 milliliters of blood are removed per kilogram of body weight of the patient.

14. The method of claim 1, wherein the type of ultraviolet radiation is selected from the group consisting of UVA, UVB, UVC, near ultraviolet, middle ultraviolet, far ultraviolet, hydrogen Lyman-alpha, vacuum ultraviolet, extreme ultraviolet, and combinations thereof.

15. The method of claim 14, wherein the type of ultraviolet radiation is selected from the group consisting of UVA, UVB, UVC, and combinations thereof.

16. The method of any preceding claim, wherein the radiation is ionizing radiation.

17. The method of any preceding claim, wherein the radiation is molecularly damaging radiation.

18. The method of any one of claims 2-13 and 16-17, wherein the radiation is particle radiation.

19. The method of claim 18, wherein the particle radiation is selected from the group consisting of alpha radiation, beta radiation, neutron radiation, positron radiation, and proton radiation.

20. The method of any preceding claim, wherein the radiation is electromagnetic radiation.

21. The method of any preceding claim, wherein the radiation has a wavelength less than about 380 nm.

22. The method of any one of claims 2-13, 16-17, and 20-21, wherein the type of radiation is ultraviolet radiation selected from the group consisting of UVA, UVB, UVC, near ultraviolet, middle ultraviolet, far ultraviolet, hydrogen Lyman-alpha, vacuum ultraviolet, extreme ultraviolet, and combinations thereof.

23. The method of claim 22, wherein the type of radiation is ultraviolet radiation selected from the group consisting of UVA, UVB, UVC, and combinations thereof.

24. The method of any one of claims 2-13 and 16-21, wherein the radiation has a wavelength less than about 10 nm.

25. The method of any one of claims 2-13 and 16-21, wherein the radiation has a wavelength less than about 10 pm.

26. The method of claim 25, wherein the radiation comprises γ-rays.

27. The method of any preceding claim, wherein the blood is exposed to radiation at a dosage sufficient to improve a treatment outcome.

28. The method of any preceding claim, wherein the treatment outcome is selected from the group consisting of reduced viral load, reduced risk of cytokine storm syndrome, reduced severity of cytokine storm syndrome, reduced risk of sepsis, reduced severity of sepsis, and reduced severity of autoimmune disease.

29. The method of claim 28, wherein the treatment outcome is reduced viral load.

30. The method of claim 29, wherein viral load in the patient is reduced by about 100%, at least about 99%, at least about 95%, at least about 90%, at least about 80%, at least about 70%, at least about 60%, at least about 50%, at least about 40%, at least about 30%, at least about 20%, or at least about 10%.

31. The method of claim 28, wherein the treatment outcome is risk of cytokine storm syndrome.

32. The method of claim 31, wherein the risk of cytokine storm syndrome in the patient after placing the exposed portion of blood into the individual is reduced by about 100%, by at least about 99%, by at least about 95%, by at least about 90%, by at least about 80%, by at least about 70%, by at least about 60%, by at least about 50%, by at least about 40%, by at least about 30%, by at least about 20%, or by at least about 10%.

33. The method of claim 28, wherein the treatment outcome is severity of cytokine storm syndrome.

34. The method of claim 33, wherein the severity of cytokine storm syndrome is determined by blood levels of a protein selected from the group consisting of an interleukin, a chemokine, a colony-stimulating factor, a tumor necrosis factor, and combinations thereof.

35. The method of any one of claims 33-34, wherein the severity of cytokine storm syndrome is determined by blood levels of a protein selected from the group consisting of IL-2, IL2R, IL-6, IL-7, IL-8, IL-10, IL-1β, IL-1RA, IL-12, IL-17, TNF-α, GSCF, IP10, MCP1/CCL2, CCL5, CXCL9, IP-10/CXCL10, MIG protein/CXCL9, MIP1A, CSF3R, IFN-y, IFNAR1, IFNGR1, CD58,TNF-α, and combinations thereof.

36. The method of claim 33, wherein the severity of cytokine storm is determined by absence of or deceased severity of a symptom selected from the group of lung injury, fever, swelling, edema, redness, fatigue, nausea, and sepsis.

37. The method of claim 28, wherein the treatment outcome is severity of autoimmune disease.

38. The method of claim 37, wherein the severity of autoimmune disease is determined by absence of or deceased severity of a symptom selected from the group of fatigue, joint pain, joint swelling, skin problems, vascular inflammation, vascular problems, myocarditis, arrhythmias, nerve problems, abdominal pain, digestive problems, fever, and swollen glands.

39. The method of any one of claims 37-38, wherein the autoimmune disease is selected from the group consisting of Crohn’s disease, eczema, psoriasis, psoriatic arthritis, rheumatoid arthritis, systemic lupus erythematosus, sarcoidosis, granulomatosis with polyangiitis, myocarditis, inflammatory bowel disease, multiple sclerosis, type 1 diabetes mellitus, Guillain-Barre syndrome, chronic inflammatory demyelinating polyneuropathy, Graves’s disease, and myasthenia graves.

40. The method of any preceding claim, wherein the patient has or had a condition selected from the group consisting of viral, bacterial, parasitic or fungal infection, acute respiratory distress syndrome, trauma, reaction to a blood transfusion, reaction to a medication, graft vs. host disease, gastric aspiration, sepsis, hemophagocytic lymphohistiocytosis, autoimmune disease, and combinations thereof.

41. The method of any one of claims 2-13 and 16-40, wherein the donor has or had a condition selected from the group consisting of viral, bacterial, parasitic or fungal infection, acute respiratory distress syndrome, trauma, reaction to a blood transfusion, reaction to a medication, graft vs. host disease, gastric aspiration, sepsis, hemophagocytic lymphohistiocytosis, autoimmune disease, and combinations thereof.

42. The method of any one of claims 40-41, wherein the condition is selected from the group consisting of viral infection, bacterial infection, parasitic infection, and fungal infection.

43. The method of claim 42, wherein the viral, bacterial, parasitic, or fungal infection is caused by an unknown microorganism.

44. The method of any preceding claim, wherein the method further comprises further treating the portion of blood.

45. The method of claim 44, wherein the further treatment comprises contacting the blood with a compound selected from riboflavin, psoralens, amotosalen HCl, porphyrins, photosensitizers, chlorophyll derivatives, light-sensitive agents, UV-activated nucleic acid replication inhibitors, dyes, coumarins, quinolones, quinones, and anthroquinones.

46. The method of any preceding claim, wherein the method causes downregulation of the patient’s immune system.

47. The method of claim 46, wherein the downregulation of the patient’s immune system is caused by inactivating, attenuating, or downregulating white blood cell activity of the patient.

48. The method of claim 46, wherein the downregulation of the patient’s immune system is caused by inactivating, attenuating, or downregulating deleterious cytokine activity in the portion of blood.

49. The method of any one of claims 5-13, and 16-48, wherein the leukocytes are selected from the group consisting of neutrophils, eosinophils, basophils, macrophages, B cells, and T cells.

50. The method of claim 49, wherein the leukocytes are T cells.

51. The method of claim 50, wherein the T cells are selected from the group consisting of CD4+ T cells, CD8+ T cells, helper T cells, cytotoxic T cells, memory T cells, regulatory T cells, natural killer T cells, mucosal associated invariant T cells, and gamma delta T cells.

52. The method of any preceding claim, wherein the radiation creates antigens in the portion of blood.

53. The method of claim 52, wherein the antigens induce an immune response in the patient.

54. The method of any of one of claims 2-13 and 16-52, wherein the patient is the donor.

55. The method of any of the preceding claims, wherein the patient has, had, or is at risk of an infection caused by at least one multi-drug resistant organism.

56. The method of any of one of claims 2-13 and 16-53, wherein the donor has, had, or is at risk of having an infection caused by at least one multi-drug resistant organism.
